(19) United States
(12) Patent Application Publication
(10) Pub. No.: US 2007/0254012 A1
(43) Pub. Date: Nov. 1, 2007

(54) Controlled degradation and drug release in stents

(76) Inventors: Florian N. Ludwig, Mountain View, CA (US); Irina Astafieva, Palo Alto, CA (US); David C. Gale, San Jose, CA (US); Syed F.A. Hossainy, Fremont, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/413,404

(22) Filed: Apr. 28, 2006

(51) Int. Cl. A61F 2/02

(52) U.S. Cl. 424/426

(57) ABSTRACT

The invention provides for a stent for implanting in a bodily lumen comprising a degradable structural element including: an abluminal layer comprising an active agent; and a luminal layer, wherein the abluminal layer has a faster degradation rate than the luminal layer.

### BACKGROUND OF THE INVENTION

1. Field of the Invention

This invention relates to stents that have controlled degradation and drug release.

2. Description of the State of the Art

This invention relates generally to implantable medical stents for treating bodily disorders. A typical treatment regimen involves implantation of a stent at a selected treatment location. During treatment, it may be necessary for the stent to support body tissue. Therefore, the structure of a stent may include load bearing structural elements or substrate to hold the stent in place and to resist forces imposed by surrounding tissue.

The treatment of a bodily disorder may also involve local delivery of a bioactive agent or drug to treat a bodily disorder. The agent may be incorporated into the stent in a variety of ways and delivered directly to an afflicted region at or adjacent to a region of implantation. An example of such a stent includes radially expandable endoprostheses, which are adapted to be implanted in a bodily lumen. An “endoprosthesis” corresponds to an artificial stent that is placed inside the body. A “lumen” refers to a cavity of a tubular organ such as a blood vessel.

A stent is an example of such an endoprosthesis. Stents are generally cylindrically shaped stents and function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The treatment of a diseased site or lesion with a stent involves both delivery and deployment of the stent. Delivery and deployment of the stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

In the case of a balloon expandable stent, the stent is mounted about a balloon disposed on the catheter. Mounting the stent typically involves compressing or crimping the stent onto the balloon. The stent is then expanded by inflating the balloon. The balloon may then be deflated and the catheter withdrawn. In the case of a self-expanding stent, the stent may be secured to the catheter via a retractable sheath or a sock. When the stent is in a desired bodily location, the sheath may be withdrawn which allows the stent to self-expand.

The stent must be capable of withstanding the structural loads, namely radial compressive forces, imposed on the stent as it supports the walls of a vessel. Therefore, a stent must possess adequate radial strength, which is the ability of a stent to resist radial compressive forces. Once expanded, the stent must adequately maintain its size and shape throughout its service life despite the various forces that may come to bear on it, including the cyclic loading induced by the beating heart. In addition, the stent must possess sufficient flexibility to allow for crimping, expansion, and cyclic loading.

The structure of a stent is typically composed of scaffolding or substrate that includes a pattern or network of interconnecting structural elements often referred to in the art as struts or bar arms. The scaffolding can be formed from wires, tubes, or sheets of material rolled into a cylindrical shape. The scaffolding is designed so that the stent can be radially compressed (to allow crimping, for example) and radially expanded (to allow deployment, for example).

Additionally, a medicated stent may be fabricated by coating the surface of either a metallic or polymeric scaffolding with a polymeric carrier that includes an active or bioactive agent or drug. Polymeric scaffolding may also serve as a carrier of an active agent or drug.

In many treatment applications, the presence of a stent in a body may be necessary for a limited period of time until its intended function of, for example, maintaining vascular patency and/or drug delivery is accomplished. Therefore, stents fabricated from biodegradable, bioabsorbable, and/or bioerodable materials such as bioabsorbable polymers can be configured to completely erode after the clinical need for them has ended. A biodegradable stent, can be fabricated so it degrades at approximately the same rate throughout its body structure. However, it may be desirable in certain treatment applications for different parts of the stent to follow different time scales of degradation.

### SUMMARY OF THE INVENTION

The invention provides for a stent for implanting in a bodily lumen comprising a degradable structural element including: an abluminal layer comprising an active agent; and a luminal layer, wherein the abluminal layer has a faster degradation rate than the luminal layer. Further, the invention provides for a stent for implanting in a bodily lumen comprising a degradable structural element including: an abluminal layer, a luminal layer, and an inner layer, the abluminal layer including an active agent, wherein the inner layer has a slower degradation rate than the abluminal and luminal layers.

Further, the invention provides for a stent for implanting in a bodily lumen comprising a degradable structural element including: an outer region above an inner region, the outer region including a first active agent and the inner region including a second active agent, wherein the inner region has a slower degradation rate than the outer region.

Further, the invention provides for a stent for implanting in a bodily lumen comprising a degradable structural element, the structural element comprising: an abluminal layer and a luminal layer, the abluminal layer having a different degradation rate than the luminal layer; and a plurality of particles configured to treat a bodily disorder releasably embedded within at least one degrading layer, wherein the particles are configured to be released from the structural element due to erosion of the at least one layer during use of the stent.

Further, the invention provides for a stent for implanting in a bodily lumen comprising a biodegradable structural element, the structural element comprising: an abluminal layer, a luminal layer, and an inner layer, the inner layer having a different degradation rate than the abluminal layer and the luminal layer; and a plurality of particles releasably embedded within at least one layer, wherein the particles are configured to be released from the structural element due to erosion of the at least one layer during use of the stent, and the plurality of particles are configured to treat a bodily disorder.

Further, the invention provides for a stent for implanting in a bodily lumen comprising a biodegradable structural element, the structural element comprising: a proximal axial segment and a distal axial segment, the proximal axial segment having a different degradation rate than the distal axial segment.

Further, the invention provides for a stent for implanting in a bodily lumen comprising a structural element, the structural element comprising: a proximal axial segment and a distal axial segment, the proximal axial segment having a different degradation rate than the distal axial segment; and plurality of particles releasably embedded within at least one segment, the particles being configured to be released from the structural element due to erosion of the at least one segment during use of the stent, the plurality of particles being configured to treat a bodily disorder.

Further, the invention provides for a stent for implanting in a bodily lumen comprising a structural element, the structural element comprising: a proximal axial segment, a distal axial segment, and an inner axial segment, the inner axial segment having a different degradation rate than the proximal and/or distal axial segment.

Further, the invention provides for a stent for implanting in a bodily lumen comprising a structural element, the structural element comprising: a proximal axial segment, a distal axial segment, and an inner axial segment, the inner axial segment having a different degradation rate than the proximal and/or distal axial segment, a plurality of particles releasably embedded within at least one segment, the particles being configured to be released from the structural element due to erosion of the at least one segment during use of the stent, the plurality of particles being configured to treat a bodily disorder.

Finally, the invention provides for a stent for implanting in a bodily lumen comprising a degradable structural element that includes an abluminal layer and a luminal layer, wherein at least one of the two layers comprise depots, the depots having a biodegradable material which at least partially fills the depots, the biodegradable material of the depots having a faster degradation rate than the layer.

### BRIEF DESCRIPTION OF THE DRAWINGS

FIG. 1 depicts a three-dimensional view of a stent.

FIG. 2 depicts a stent mounted on a catheter within a vascular segment.

FIG. 3 depicts a stent implanted in a vascular segment.

FIG. 4A depicts a two-dimensional view of a side-wall of a segment of a strut.

FIG. 4B depicts a close-up view of a portion of the strut segment in FIG. 4A.

FIG. 5A depicts a section of stent from FIG. 1 with an abluminal layer and a luminal layer.

FIG. 5B depicts a tube with an abluminal layer and a luminal layer.

FIG. 6A depicts a section of a stent having particles releasably embedded within an abluminal layer and a luminal layer.

FIG. 6B depicts a section of a stent having depots on the surface of the abluminal layer for carrying an active agent.

FIG. 7A depicts a section of a stent having an abluminal layer, a luminal layer, and an inner layer.

FIG. 7B depicts a section of a stent having an outer region and an inner region that has a slower degradation rate than the outer region.

FIG. 8A depicts a section of a stent having an abluminal layer and a luminal layer, with particles in the abluminal layer and particles in the abluminal layer.

FIG. 8B depicts a section of a stent having an abluminal layer and a luminal layer, and particles in the luminal layer.

FIG. 9 depicts a section of a stent having an abluminal layer, a luminal layer, and an inner layer, and particles in the abluminal layer and the luminal layer.

FIG. 10 depicts a stent having a proximal axial segment and a distal axial segment.

FIG. 11 depicts a stent having an inner axial segment between a proximal axial segment and a distal axial segment.

FIG. 12 depicts a stent having a proximal axial segment and a distal axial segment, and particles in the proximal axial segment and the distal axial segment.

FIG. 13 depicts a stent having a proximal axial segment, an inner axial segment, and a distal axial segment, and particles in each segment.

### DETAILED DESCRIPTION OF THE INVENTION

In general, treatment of a bodily disorder with an implantable medical device, such as a stent, has several functional requirements. A stent provides structural support to the body tissue in which it is implanted, in which case the stent must have a structural pattern that is compatible with the body tissue in which it is implanted. In addition, a stent may deliver a bioactive agent to an implanted region for treatment of a bodily disorder. It may also be desirable for the stent to disintegrate and disappear from the implanted region once treatment is complete.

Various embodiments of the present invention relate to stents for treating bodily tissue disorders local and distal to the region that the stent is implanted. The stent may be configured to disintegrate and disappear from the region that the stent is implanted once treatment is completed.

The term “stent” includes, but is not limited to, self-expandable stents, balloon-expandable stents, stent-grafts, urethral stents, and pulmonary stents.

For the purposes of the present invention, the following terms and definitions apply:

“Bodily disorder” refers to any condition that adversely affects the function of the body.

“Dissolve” refers to a substance passing into solution on a molecular scale with or without chemical breakdown of the substance.

The term “treatment” includes prevention, reduction, delay, stabilization, or elimination of a bodily tissue disorder, such as a vascular disorder. In some embodiments, treatment also includes repairing damage caused by the disorder and/or mechanical intervention.

“Use” includes stent delivery to a treatment site and stent deployment or implantation at a treatment site.

A “bioactive” or “active” agent can be any substance capable of exerting an effect including, but not limited to, therapeutic, prophylactic, or diagnostic. Bioactive agents may include anti-inflammatory and antiproliferative and other bioactive agents.

In general, the structure of a stent includes structural elements, scaffolding, or a substrate that may be the primary source of structural support. For example, a stent typically is composed of a pattern or network of circumferential rings and longitudinally extending interconnecting structural elements of struts or bar arms. In general, the struts are arranged in patterns, which are designed to contact the lumen walls of a vessel and to maintain vascular patency.

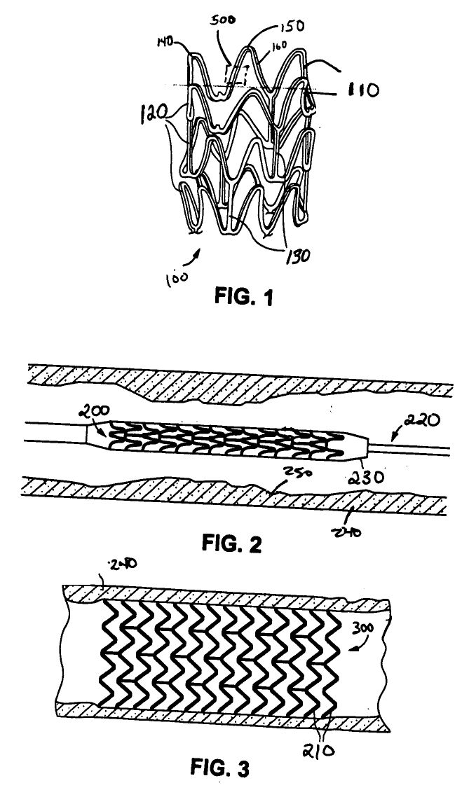

FIG. 1 depicts a three-dimensional view of a stent 100 which is made up of struts 110. Stent 100 has interconnected cylindrical rings 120 connected by linking struts or links 130. The struts of stent 100 have a luminal surface 140, abluminal surface 150, and sidewall surfaces 160. In some embodiments, the diameter of the stent can be between about 0.2 mm and about 5.0 mm, or more narrowly between about 1 mm and about 3 mm. Unless otherwise specified, the “diameter” of the stent refers to the outside diameter of tube.

Conventionally, a stent such as stent 100 may be fabricated from a tube by forming a pattern with a technique such as laser cutting. The embodiments disclosed herein are not limited to stents or to the stent pattern depicted in FIG. 1.

FIGS. 2-3 illustrate local treatment of diseased sites in a bodily lumen with a stent. FIGS. 2-3 can represent any balloon expandable stent 200 with which various configurations can be used. The explanation below can easily be adapted to a self-expandable stent. FIG. 2 depicts a stent 200 with interconnected cylindrical rings 210 mounted on a catheter assembly 220. Catheter assembly is used to deliver stent 200 and implant it into a bodily lumen. The catheter assembly is configured to advance through the patient's vascular system by advancing over a guide wire by any methods known in the art. The stent is mounted on an expandable member 230 (e.g., a balloon) and is crimped tightly so that the stent and expandable member present a low profile diameter for delivery through the arteries.

As shown in FIG. 2, a partial cross-section of an artery 240 has a diseased area or lesion 250. Stent 200 is used to repair a diseased or damaged arterial wall as shown in FIG. 2, or a dissection, or a flap, all of which are commonly found in the coronary arteries and other vessels. Stent 200 and other embodiments of stents can also be placed and implanted without any prior angioplasty.

In a typical procedure to implant stent 200, catheter assembly 220 is advanced through the patient's vascular system by well-known methods to diseased area 250. The expandable member or balloon 230 is inflated by well-known means so that it expands radially outwardly and in turn expands the stent radially outwardly until the stent is opposed to the vessel wall. The expandable member is then deflated and the catheter withdrawn from the patient's vascular system. In FIG. 3, implanted stent 300 remains in the vessel after the balloon has been deflated and the catheter assembly and guide wire have been withdrawn from the patient. Stent 300 holds open the artery after the catheter is withdrawn, as illustrated by FIG. 3.

Some treatments with stents require the presence of the stent only for a limited period of time. The duration of a treatment period depends on the bodily disorder that is being treated. Once treatment is complete, the stent is removed or disappears from the treatment location. One way of having a stent disappear may be by fabricating the stent in whole or in part from materials that erode or disintegrate through exposure to conditions within the body.

In general, polymers can be biostable, bioabsorbable, biodegradable, or bioerodable. Biostable refers to polymers that are not biodegradable. The terms biodegradable, bioabsorbable, and bioerodable, as well as degraded, eroded, and absorbed, are used interchangeably and refer to polymers that are capable of being completely eroded or absorbed after implantation, e.g., when exposed to bodily fluids such as blood and can be gradually resorbed, absorbed, and/or eliminated by the body.

As indicated above, a stent can be medicated for treating a bodily disorder at or adjacent to an implant region. A stent can be medicated in a number of ways. First, as mentioned above, a biodegradable stent, may be fabricated by coating the surface of a polymeric scaffolding to produce a drug reservoir layer on the surface. The drug reservoir layer typically includes a polymeric carrier that includes an active agent or drug. To fabricate a conventional coating, a polymer or a blend of polymers can be applied on the stent using techniques known to those having ordinary skill in the art. The coating may be applied to the stent, for example, by immersing the stent in a coating material including a polymer, solvent, and active agent or by spraying the coating material onto the stent.

Also, as indicated above, all or part of a polymeric scaffolding of a stent may also serve as a carrier of an active agent or drug. The active agent or drug can be incorporated into the scaffolding during fabrication of the stent. In one embodiment, an active agent can be dispersed within a polymer during extrusion of tube, from which a stent can be fabricated. In another embodiment, the active agent may be disposed in depots at a surface of the stent.

Further, all or part of a stent can be fabricated from a plurality of drug-loaded particles releasably embedded in a biodegradable polymeric matrix. Alternatively, a scaffolding of a stent can have coating of drug-loaded particles releasably embedded in the biodegradable polymeric matrix. Once a stent is implanted, the polymeric matrix erodes, allowing particles to be released from the stent.

The use of releasably embedded particles in a stent allows treatment of bodily disorders distal from the implant region. Particles may also be disposed in depots at a surface of the stent. Bodily tissue disorders may be treated with an active agent locally. Local treatment refers to administration of an active agent at or adjacent to the bodily tissue disorder. For example, stents may provide for the local administration or delivery of an active agent at a diseased site at or adjacent to the region that the stent was implanted.

In some treatment situations, local treatment of bodily tissue disorders with a stent may be difficult or impossible. This inability may be due to the fact that tissue disorders may be diffuse and in multiple locations. Local treatment in such situations may require a number of stents. For example, vascular disorders can include lesions in multiple locations, such as diffuse lesions along vessels, multi-vessel lesions, and bifurcated vessel lesions. In addition, local treatment may be impossible because an afflicted region of tissue may be inaccessible to implantation of a stent. For example, a diseased vessel may be too small for implantation of a stent. However, drug-loaded particles released from stent can be transported to regions distal from the implant region allowing treatment of bodily disorders in such regions.

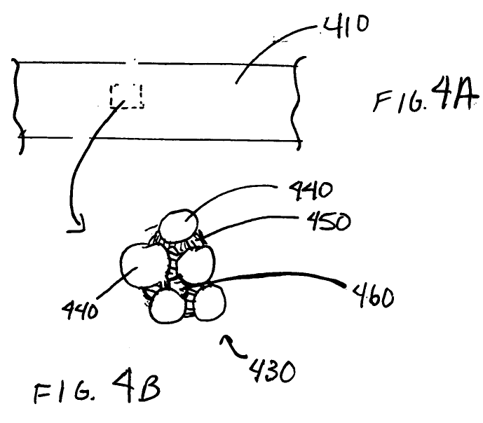

FIGS. 4A and 4B depict an example of a strut from a stent including particles bound together with a biodegradable material according to an embodiment described above. FIG. 4A depicts a two-dimensional view of a sidewall of a segment of a strut 410. FIG. 4B shows a close-up view of a portion 430 of strut 410. Portion 430 has particles 440 bound together by biodegradable material 450. The structure of strut 410 may also include cavities or pores 460. In one embodiment, particles 440 include an active agent. Thus, particles 440 may deliver the active agent to a region for treatment of a disorder by eluting from the particles to treat the disorder.

According to the invention, drug release from a stent structure can be controlled by degradation. As a biodegradable polymer degrades or is absorbed into the body, a drug incorporated into the stent may be simultaneously released from the stent. First, in the case of a drug-impregnated coating or substrate, the degradation or absorption rate of the coating or substrate polymer can be greater than the diffusion rate of the drug through the polymer and out of the stent. Second, in the case of drug-loaded particles embedded in a polymer matrix, the release rate of the particles is governed by degradation of the polymer matrix. Thus, drug release in both cases tends to follow degradation kinetics of the polymer. It follows that drug release kinetics can be tuned or controlled by degradation rate of a coating, substrate, or polymer matrix.

The present invention provides for a stent having different rates of degradation at different locations within the stent. Accordingly, the drug release from the stent depends on the location of the drug in the stent. A stent having a spatially varying degradation rate can be advantageous in a numerous treatment situations. For example, a portion of a stent may have a slower degradation rate for maintaining structural integrity of the stent while drug is released from a faster degrading portion. Also, the drug may be preferentially released to treat selected afflicted tissue. In addition, different drugs may be released over different time frames. For example, for stents that are intended to release multiple pharmaceutical agents, active agents may need to be released over different time frames, such as in treatment of different conditions or different aspects of the same condition. For instance, a lesion with an anti-inflammatory drug may need to be treated initially, followed by treatment with an antiproliferative drug. A person of skill in the art can appreciate numerous other situations in which a spatially varying degradation rate can be advantageous.

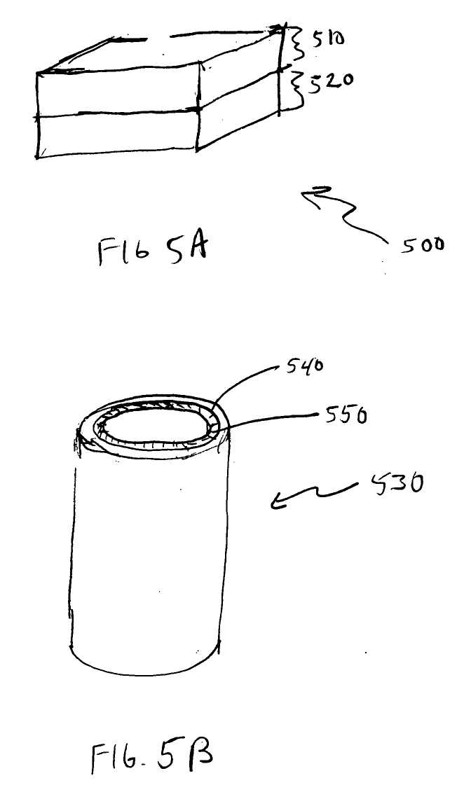

The degradation rate in a stent can vary spatially in many different ways. In some embodiments, a stent can have struts with a radially varying degradation rate. Specifically, a degradation rate profile along a radial coordinate can vary from an abluminal surface to a luminal surface of a strut. For example, various embodiments may include a stent having struts with radial layers in which at least two layers have different degradation rates. FIG. 5A depicts a section 500 of stent 100 from FIG. 1 with an abluminal layer 510 and a luminal layer 520. Abluminal layer 510 and luminal layer 520 can be composed of biodegradable materials having different degradation rates. Either abluminal layer 510 or luminal layer 520 can degrade faster. The strut may also include one or more middle or inner layers between abluminal layer 510 and luminal layer 520. A degradation profile can include, for example, slower degrading outer layers (luminal, abluminal) and faster degrading inner layers.

A stent having radial layers may be formed, for example, from a layered tube. FIG. 5B depicts a tube 530 with an outer layer 540 (corresponding to abluminal layer 510) and an inner layer 550 (corresponding to luminal layer 520). Tube 530 can be formed by, for example, coextrusion of two biodegradable materials with different degradation rates, which can be of two different biodegradable polymers. A stent can be formed from the tube by forming a pattern in the tube by laser cutting or chemical etching, for example.

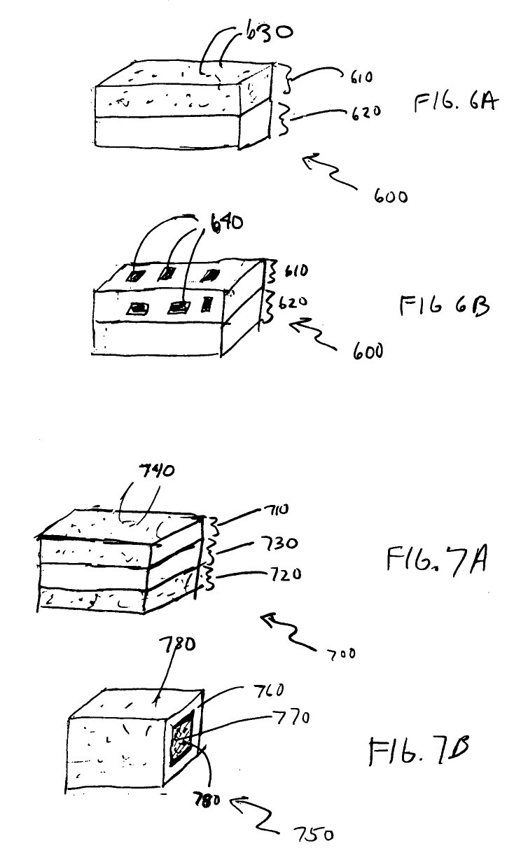

In some embodiments, a stent has a degradable structural element including a luminal layer and an abluminal layer. At least one layer can include an active agent. In one embodiment, the abluminal layer has a faster degradation rate than the luminal layer. FIG. 6A depicts a section 600 of a stent with an abluminal layer 610 and a luminal layer 620.

As depicted in FIG. 6A, active agent 630 is dispersed in abluminal layer 610. Active agent 630 may be mixed or dispersed within abluminal layer 610, luminal layer 620, or both luminal and abluminal layer.

In one embodiment, abluminal layer 610, luminal layer 620, or both abluminal layer 610 and luminal layer 620 comprise depots. The depots have a biodegradable material that at least partially fills the depots, and the biodegradable material of the depots having a faster degradation rate than the layer. As depicted in FIG. 6B, abluminal layer 610 can include depots 640. In one embodiment, the depots include an active agent configured to treat a disorder. In another embodiment, the depots contain particles. The particles disposed in the depots can also include an active agent configured to treat a disorder. As depicted in FIG. 6B, active agent 630 can also be disposed in depots 640 on the surface of abluminal layer 610.

Abluminal layer 610 can be composed of a biodegradable material that has a higher degradation rate than a biodegradable material of luminal layer 620. During treatment, abluminal layer 610 can release active agent 630 into the vessel tissue. Since luminal layer 620 is slower degrading, the luminal layer provides structural integrity to the stent and support the lumen during active agent release from the abluminal layer. When release is complete, structural support of the stent may no longer be needed and luminal layer 620 may disintegrate. In other embodiments, abluminal layer 610 is slower degrading, and luminal layer 620 is faster degrading. For example, luminal layer may contain an active agent that is released from the faster degrading biodegradable material, and the abluminal layer can provide structural support of the lumen.

Additionally, a stent may also be used to deliver multiple active agents that can be released in different time frames. Luminal layer 620 may also have an active agent mixed or dispersed within or have depots with active agent. Since abluminal layer 610 is faster degrading, the active agent within the abluminal layer 610 will be released faster than active agent 630 in luminal layer 620. In one embodiment, an anti-inflammatory active agent is incorporated into faster degrading abluminal layer 610 and an antiproliferative active agent is incorporated into slower degrading luminal layer 620.

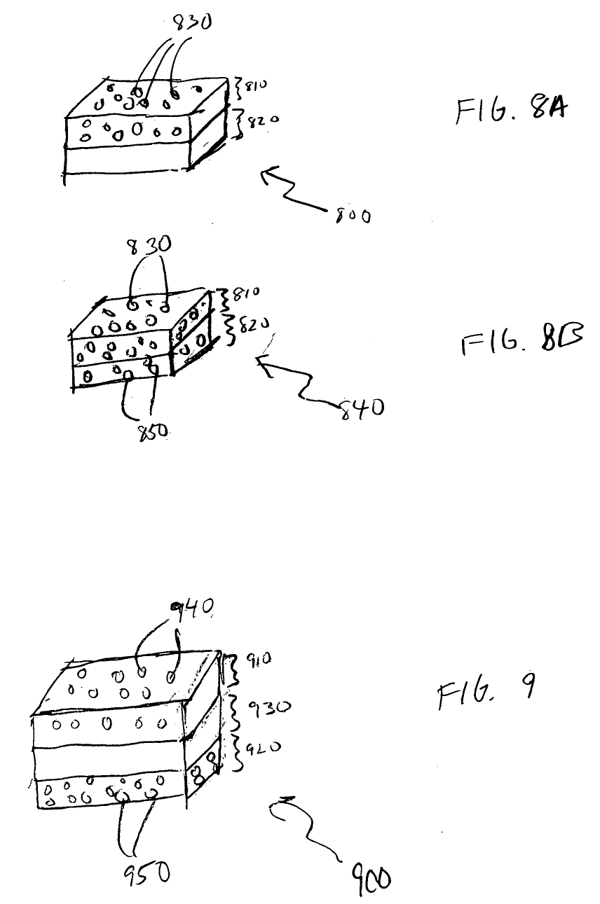

FIG. 7A depicts a section 700 of a stent which has an abluminal layer 710, a luminal layer 720, and an inner layer 730, at least one layer having a slower degradation rate than another layer. For example, inner layer 730 may have a slower degradation rate than abluminal layer 710 and luminal layer 720. Abluminal layer 710 and/or luminal layer 740 may include an active agent 740. Inner layer 730 can have a slower degradation rate than abluminal layer 710 and luminal layer 720. Inner layer 720 can provide the structural integrity to the stent to support the lumen during the release of active agent 740 from abluminal layer 710 and also from luminal layer 720. Additionally, inner layer 720 can also include an active agent which can be the same or different from active agent 740. For instance, an anti-inflammatory active agent can be incorporated into the faster degrading abluminal layer 710 and luminal layer 720, and an antiproliferative active agent can be incorporated into the slower degrading inner layer 730.

Another embodiment of a stent can have a degradable structural element with an outer region above an inner region with the outer region including a first active agent and the inner region including a second active agent. The inner region can have a slower degradation rate then the outer region. Such an embodiment may allow release of different active agents during different time frames. FIG. 7B depicts a section 750 of a stent which has an outer region 760 and an inner region 770 that has a slower degradation rate than outer region 760. Outer region 760 includes an active agent 780 and inner region 770 has an active agent 790 that is different from active agent 780. Inner region 770 can provide the structural integrity to the stent during release of active agent 780 from outer region 760. As above, an anti-inflammatory active agent may be incorporated into the faster degrading outer region 760, and an antiproliferative active agent may be incorporated in the slower degrading inner region 770.

Inner region 770, for example, can be in a substrate or scaffolding of a stent, and outer region 760 can be a coating. In another embodiment, the structural element can be a fiber formed by coextruding different biodegradable materials.

In other embodiments, a degradable structural element includes an abluminal layer and a luminal layer having a different degradation rate and a plurality of particles releasably embedded within at least a layer. In one embodiment, the particles may be embedded in a faster degrading layer. The particles may include active agents within the particles for treating a bodily disorder. A layer without particles can also have active agents mixed or dispersed within the biodegradable material.

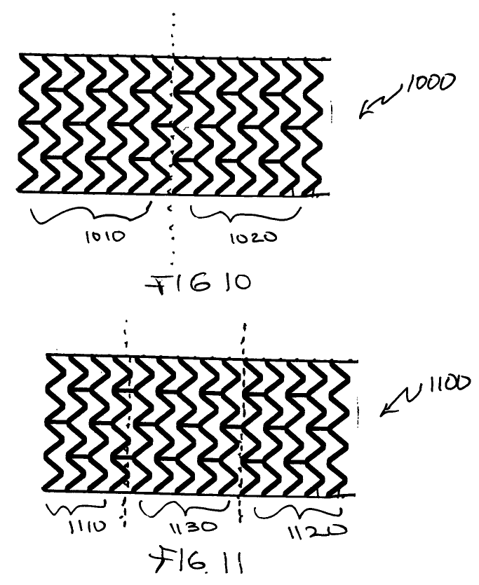

Erosion of the layers may allow at least some of the particles to be released from the structural element of the stent. FIG. 8A depicts a section 800 of a structural element having an abluminal layer 810 and a luminal layer 820 composed of biodegradable materials having different degradation rates. Particles 830 are shown to be embedded in the abluminal layer. Particles 830 can include an active agent that is released from particles 830 to treat a bodily disorder. Particles 830, however, need not carry an active agent. In an embodiment, one or more of the particles can be disposed in depots situated in the layers. Erosion of a faster degrading abluminal layer 810, for example, allows particles 830 to be released into afflicted tissue at the vessel wall.

Abluminal layer 820 can include an active agent or drug-loaded particles in a slower degrading matrix. Particles from the abluminal, fast-degrading layer 810 can be released into the tissue faster than the release of the active agent from the luminal layer 810. As above, slower degrading luminal layer 820 can provide structural integrity to the stent in supporting the lumen during active agent release from abluminal layer 810.

As depicted in FIG. 8B, luminal layer 820 can have particles 850 that can be released into the lumen as luminal layer 850 erodes. Particles 850 can be of the same or different type of active agent than particles 850 in luminal layer 830. As indicated above, particles 850 can be transported to regions distal to the implant region. Active agent within particles 850 can then treat bodily disorders in such distal regions, as well as disorders local to the implant region.

The particles may be arranged in the layers such that selected particles are released over different times dictated by a treatment regimen. For example, particles 830 within fast degrading abluminal layer 810 may include an anti-inflammatory agent and particles 850 in slower degrading luminal layer 820 may include an antiproliferative agent. In another embodiment, a spatially varying degradation rate can be used to vary the dose of active agent with time. For example, a heavy dose may be required initially but a light dose may follow. To vary treatment dosage with time, drug loading of the particles can be made to vary in the layers.

In another embodiment, a stent can have a structural element with an inner layer that has a different degradation rate than an abluminal layer and a luminal layer. At least one of the layers may have particles releasably embedded within. FIG. 9 depicts a section 900 of a structural element of a stent having an abluminal layer 910, luminal layer 920, and an inner layer 930. In FIG. 9, particles 940 and 950 are shown to be releasably embedded in layers 910 and 920, respectively. Particles 940 and 950 may be devoid of, have no active agent, or have the same active agent, or different active agents.

In one embodiment, inner layer 930 can have a slower degradation rate than abluminal layer 910 and luminal layer 920. In this case, inner layer 930 provides structural integrity to the structural element as the outer layers degrade. Inner layer 930 can also be made to be faster degrading than abluminal layer 910 and luminal layer 920. Additionally, inner layer 930 can have a degradation rate between abluminal layer 910 and luminal layer 920 so that the degradation rate increases or decreases from the abluminal to luminal surface. The structural element is not limited to one inner layer as depicted in FIG. 9, as there can be multiple inner layers of the same or different biodegradable material, and the same or different degradation rates.

Furthermore, various embodiments of a stent can also be made such that the degradation rate varies axially or longitudinally along a stent. FIG. 10 depicts a stent having a proximal axial segment 1010 and a distal axial segment 1020. In one embodiment, at least a portion of proximal axial segment 1010 has a faster degradation rate compared to distal axial segment 1020. Alternatively, at least a portion of distal axial segment 1020 has a faster degradation rate compared to proximal axial segment 1010. Although axial “segments” are depicted as being the entire circumference of the stent, it should be understood that only portions of the axial segments can vary in degradation rate. For example, proximal axial “segment” can be 20% of the circumference of the stent.

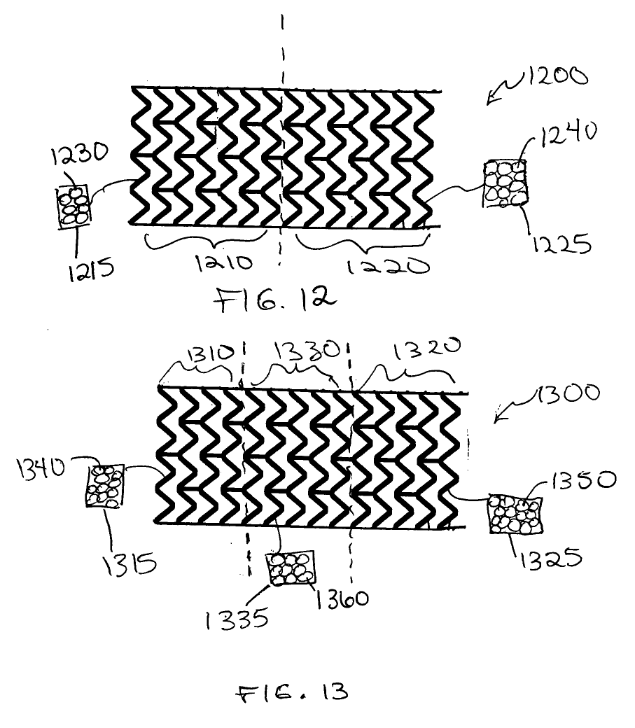

Additionally, a proximal axial segment and a distal axial segment of a stent may have a different degradation rate as compared to an inner axial segment. As depicted in FIG. 11, stent 1100 has an inner axial segment 1130 between proximal axial segment 1110 and distal axial segment 1120. Proximal axial segment 1110 and distal axial segment 1120 can have a different degradation rate compared to inner axial segment 1130. The relative degradation rates and length of the segments depend on the desired application of the stent. For example, proximal axial segment 1110 and distal axial segment 1120 can have a faster or slower degradation rate than inner axial segment 1130. Alternatively, inner axial segment 1130 can have a degradation rate between proximal axial segment 1110 and distal axial segment 1120. It should be understood by those skilled in the art that a stent can have multiple axial segments with different degradation rates. In one embodiment, one or more of the axial segments can be coated to obtain a different degradation rate as compared to other axial segments.

An inner axial segment 1130 having a faster degradation than proximal axial segment 1110 and distal axial segment 1120, for example, can be useful in providing a faster active agent release from the inner portion of the stent in relation to the proximal and distal portions of a stent. For example, a lesion may be more pronounced adjacent to an inner axial segment 1130, and thus, a faster drug release of the inner axial segment of a stent may be needed.

In one embodiment, a proximal axial segment 1110 and the distal axial segment 1120, where distal axial segment 1120 has a faster degradation, for example, can be used to maintain support of the lumen, while also providing flexibility with a slower degrading inner axial segment 1130. Thus, axial segments having different degradation rates can also exhibit different mechanical properties. A stent having axial segments with different degradation rates exhibits more flexibility. The increase in flexibility may be more significant when axial segments alternate in relative degradation rates. A greater flexibility can facilitate delivery of the stent. Furthermore, degradation causes changes in mechanical properties. For example, as a stent degrades, the difference in mechanical properties can become more pronounced.

In some embodiments, particles can be releasably embedded within a stent having a degradation rate that varies longitudinally along the stent. The particles can be drug-loaded, such that a drug is released upon degradation of the stent to treat bodily disorders in local and/or distal regions to the implant region. As depicted in FIG. 12, stent 1200 includes proximal axial segment 1210 having a different degradation rate than distal axial segment 1220. Further, stent 1200 includes a plurality of particles 1230 releasably embedded within proximal axial segment 1210 and particles 1240 in distal axial segment 1220 as shown in blown up portions 1215 and 1225. Particles can be loaded with the same or different active agent. In one embodiment, distal axial segment 1220 can have a faster degradation rate than proximal axial segment 1210, so particles 1240 of distal axial segment 1220 can be released before particles 1230 of proximal axial segment 1210. For example, particles 1240 of distal axial segment 1220 may be loaded with an anti-inflammatory drug and particles 1230 of proximal axial segment 1210 may be loaded with an anti-proliferative. Particles can treat a disorder local to the implant region or a disorder downstream of the stent. In general, particles can be arranged in the axial segments such that selected particles are released from the faster eroding segments before those in slower eroding segments.

FIG. 13 depicts a stent 1300 with a proximal axial segment 1310, a distal axial segment 1320, and an inner axial segment 1330. Stent 1300 includes particles 1340, 1350, 1360 releasably embedded within at least one axial segment that is configured to treat a bodily disorder as shown in blown up portions 1315, 1325, and 1335, respectively. Particles 1340, 1350, 1360, respectively are configured to be released from stent 1300 due to erosion of the at least one of the segments during use of the stent.

In one embodiment, inner axial segment 1330 has a faster degradation rate than the proximal axial segment 1310 and the distal axial segment 1320, allowing a majority of particles 1350 in inner axial segment 1330 to be released before a majority of particles in the proximal axial segment 1310 and distal axial segment 1320. In another embodiment, inner axial segment 1330 has a slower degradation rate than proximal axial segment 1310 and distal axial segment 1320 which allows a majority of particles 1360 in distal axial segment 1320 and/or a majority of particles 1340 in proximal axial segment 1310 to be released before a majority of particles 1350 in inner axial segment 1330.

Several mechanisms may cause erosion and disintegration of stents which include, but are not limited to, mechanical, chemical breakdown, dissolution, and breakdown due to rheological forces. Therefore, bodily conditions can include, but are not limited to, all conditions associated with bodily fluids (contact with fluids, flow of fluids) and mechanical forces arising from body tissue in direct and indirect contact with a stent. Chemical breakdown of biodegradable materials results in changes of physical and chemical properties of the polymer, for example, following exposure to bodily fluids in a vascular environment. The changes in properties may include a decrease in molecular weight, deterioration of mechanical properties, and decrease in mass due to erosion or absorption.

Chemical breakdown includes hydrolysis. In general, hydrolysis is a chemical process in which a molecule is cleaved into two parts by the addition of a molecule of water. With respect to a bioabsorbable polymer such as PLLA, water takes part in the hydrolysis of ester bonds in the polymer backbone which leads to the formation of water-soluble fragments. Consequently, the rate of degradation of a biodegradable polymer is strongly dependent on the concentration of water in the polymer. A higher concentration of water in a polymer can lead to a faster rate of hydrolysis, tending to result in a shorter degradation time of a device made from the polymer.

Several characteristics or parameters of the degradation process are important in designing biodegradable stents, including an average erosion rate of a stent, erosion profile, half-life of the degrading polymer, and mechanical stability of a stent during the degradation process. The “average erosion rate” may be an average erosion rate over any selected time interval:
Average erosion rate=(m₁−m₂)/(t₂−t₁)
where “m” refers to mass of the stent, “t” refers to a time during erosion, and m₁ and m₂ are the masses of the stent at t₁ and t₂ during erosion. For instance, the selected time interval may be between the onset of degradation and another selected time. Other selected times, for example, may be the time for about 25%, 50%, 75%, or 100% (complete erosion) of the stent to erode. Complete erosion may correspond approximately to the time required for treatment by the stent. As an example of the time frame of erosion, a biodegradable stent may be completely eroded in about six to eighteen months.

The “half-life” of a degrading polymer refers to the length of time for the molecular weight of the polymer to fall to one half of its original value. See e.g., J. C. Middleton and A. J. Tipton, Biomaterials, Vol. 21 (23) (2000) pp. 2335-2346.

Various properties of a polymeric material may be used to vary the rate of degradation or erosion and release of particles. Thus, a variation of such properties in layers or axial segments of a stent can be used to change the degradation rates in the layers or axial segments. In general, erosion rate depends on a number of factors including, but not limited to, chemical composition, porosity, molecular weight, and degree of crystallinity. A higher porosity may increases the erosion rate. Molecular weight tends to be inversely proportional to degradation rate. Also, a higher degree of crystallinity tends to result in a lower degradation rate. Thus, amorphous regions of a polymer can have a higher degradation rate than crystalline regions. Additionally, the chemical make-up of a polymer also effects the erosion rate of the polymer.

In some embodiments, spatially varying degradation in a stent can be induced through the use of regioselective thermal processing. In regioselective thermal processing, a selected portion of the body structure is selectively heated, thereby lowering the molecular weight of that portion of the polymer. Lowering the molecular weight of the polymer in the selected regions also increases the degradation rate of the polymer. For example, selected axial portions of a stent can be selectively heated to change the degradation rate of the selected axial portions.

In yet another embodiment, the spatially varying degradation rate can be imparted in a stent by fabricating the stent from a tube with a gradient of hydrophilic compounds. By incorporating hydrophilic compounds with a polymer layer, the level of moisture within the polymer is increased. In general, the rate of hydrolysis of a polymer is a function of the concentration of water in the polymer stent. Higher levels of moisture in the structural elements of a stent lead to a faster rate of hydrolysis of the element, resulting in a shorter degradation time for the stent. The degradation rate then becomes controlled by degree of water uptake. Such hydrophilic compounds include, but are not limited to, high molecular weight poly(ethylene oxide), poly(vinyl pyrrolidone), etc. In one embodiment, the gradient in degradation rate can be formed in a stent by forming the stent from a coextruded tube in which at least one layer has hydrophilic compounds.

In another embodiment, the body structure of the stent can be formed by impregnating absorption initiators in a gradient fashion. Absorption initiators can be incorporated into selected layers or selectively coated on a stent. For example, dilactide monomers can be used as absorption initiators in a polylactide stent. The absorption initiator sin a layer or coating can induce a concentration gradient which will create a gradient in absorption rate.

In one embodiment, stereolithography or patterned lithography may be used to impart differential degradation in a stent. “Stereolithography” or “3-D printing” or “patterned lithography” refers to a technique for manufacturing solid objects by the sequential delivery of energy and/or material to specified points in space to produce that solid. The manufacturing process may be controlled by a computer using a mathematical model created with the aid of a computer. A coating material including particles may be applied to a stent by an applicator, such as a nozzle, programmed to apply the material in a pattern corresponding to the predefined portion of particles. The pattern may be based on computer-generated construct of the stent.

A stent may be made from a material including, but not limited to, bioabsorbable polymer; a biosoluble material; a biopolymer; a biostable metal; a biodegradable metal; a block copolymer of a bioabsorbable polymer or a biopolymer; a bioabsorbable ceramic; or a combination thereof. The erosion of the material can be due to dissolution, chemical breakdown, and/or enzymatic degradation of the polymer material and/or particles.

Representative examples of polymers that may be used to fabricate embodiments of stents, coatings for stents, and particles disclosed herein include, but are not limited to, poly(N-acetylglucosamine) (Chitin), Chitosan, poly(3-hydroxyvalerate), poly(lactide-co-glycolide), poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, biomolecules (such as fibrin, fibrin glue, fibrinogen, cellulose, starch, collagen and hyaluronic acid, elastin and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. Additional representative examples of polymers that may be especially well suited for use in fabricating embodiments of stents disclosed herein include ethylene vinyl alcohol copolymer (commonly known by the generic name EVOH or by the trade name EVAL), poly(butyl methacrylate), poly(vinylidene fluoride-co-hexafluoropropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, poly(vinyl acetate), styrene-isobutylene-styrene triblock copolymers, and polyethylene glycol.

Representative examples of biosoluble materials that may be used to fabricate embodiments of stents, coatings for stents, and particles disclosed herein include, but are not limited to, poly (ethylene oxide); poly (acrylamide); poly (vinyl alcohol); cellulose acetate; blends of biosoluble polymer with bioabsorbable and/or biostable polymers; N-(2-hydroxypropyl) methacrylamide; and ceramic matrix composites.

The stent can also be fabricated from erodible metals. Metals may be biostable or bioerodible. Some metals are considered bioerodible since they tend to erode or corrode relatively rapidly when implanted or when exposed to bodily fluids. Biostable metals refer to metals that are not bioerodible. Biostable metals have negligible erosion or corrosion rates when implanted or when exposed to bodily fluids. Representative examples of biodegradable metals that may be used to fabricate a stent may include, but are not limited to, magnesium, zinc, and iron.

Embodiments of the stent can include numerous types and configurations of particles; Representative examples of materials that may be used for particles include, but are not limited to, a biostable polymer; a bioabsorbable polymer; a biosoluble material; a biopolymer; a biostable metal; a bioerodible metal; a block copolymer of a bioabsorbable polymer or a biopolymer; a ceramic material such as a bioabsorbable glass; salts; fullerenes; lipids; carbon nanotubes; or a combination thereof. Particles may also include micelles or vesicles.

Particles may have bioactive agents mixed, dispersed, or dissolved in the particle material. Particles may also be coated with an active agent. In other embodiments, particles can also have an outer shell of polymer, metal, or ceramic with inner compartment containing an active agent. In an embodiment, particles may include bioabsorbable glass with bioactive agent encapsulating or embedded within the particle. In some embodiments, particles may be designed to use a combination of the above, e.g., a particle may include a polymeric drug, or a drug impregnated core coated with a bioerodible metal. In addition, particles may include fullerenes coated with a bioactive agent.

In certain embodiments, the particles may include nanoparticles and/or microparticles. A nanoparticle refers to a particle with a characteristic length (e.g., diameter) in the range of about 1 nm to about 1,000 nm. A microparticle refers to a particle with a characteristic length in the range of greater than 1,000 nm and less than about 10 micrometers.

As discussed above, the particles may have different treatment properties. The treatment properties that the active agent in the particles may have include, but are not limited to, type(s) of active agent included in each particle, release rate of active agents from the particle, degradation rate, and size. Some particles may have different types of active agents, different release rates than other particles, different degradation rates, and different sizes.

As indicated above, the particles and the biodegradable material may include active agent(s) such as anti-inflammatories, antiproliferatives, and other bioactive agents. An antiproliferative agent can be a natural proteineous agent such as a cytotoxin or a synthetic molecule. Preferably, the active agents include antiproliferative substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin D include dactinomycin, actinomycin IV, actinomycin II, actinomycin X₁, and actinomycin C₁), all taxoids such as taxols, docetaxel, and paclitaxel, paclitaxel derivatives, all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy] ethyl-rapamycin, or 40-O-tetrazole-rapamycin, 40-epi-(N1-tetrazolyl)-rapamycin (ABT-578 manufactured by Abbot Laboratories, Abbot Park, Ill.), prodrugs thereof, co-drugs thereof, and combinations thereof. In one embodiment, the anti-proliferative agent is everolimus.

An anti-inflammatory drug can be a steroidal anti-inflammatory agent, a nonsteroidal anti-inflammatory agent, or a combination thereof. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof. In one embodiment, the anti-inflammatory agent is clobetasol.

Alternatively, the anti-inflammatory may be a biological inhibitor of proinflammatory signaling molecules. Anti-inflammatory biological agents include antibodies to such biological inflammatory signaling molecules.

In addition, the particles and biodegradable material may include agents other than antiproliferative agent or anti-inflammatory agents. These active agents can be any agent which is a therapeutic, prophylactic, or a diagnostic agent. In some embodiments, such agents may be used in combination with antiproliferative or anti-inflammatory agents. These agents can also have anti-proliferative and/or anti-inflammmatory properties or can have other properties such as antineoplastic, antiplatelet, anti-coagulant, anti-fibrin, antithrombonic, antimitotic, antibiotic, antiallergic, antioxidant, and cystostatic agents. Other bioactive agents may include antiinfectives such as antiviral agents; analgesics and analgesic combinations; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary; peripheral and cerebral; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and restenoic reducing agents. The foregoing active agents are listed by way of example and are not meant to be limiting. Other active agents which are currently available or that may be developed in the future are equally applicable.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A stent for implanting in a bodily lumen comprising a degradable structural element including:
an abluminal layer comprising an active agent; and
a luminal layer, wherein the abluminal layer has a faster degradation rate than the luminal layer.

2. The stent according to claim 1, wherein the luminal layer comprises a second active agent.

3. The stent according to claim 2, wherein one of the active agents is an anti-inflammatory agent and the other active agent is an antiproliferative agent.

4. A stent for implanting in a bodily lumen comprising a degradable structural element including:
an abluminal layer, a luminal layer, and an inner layer, the abluminal layer including an active agent, wherein the inner layer has a slower degradation rate than the abluminal and luminal layers.

5. The stent according to claim 4, wherein the inner layer further comprises a second active agent.

6. The stent according to claim 4, wherein luminal layer further comprises an active agent.

7. The stent according to claim 5, wherein the active agent is selected from the group consisting of anti-proliferative agent and anti-inflammatory agent.

8. The stent according to claim 5, wherein one of the active agents is an anti-inflammatory agent and the other agent is an anti-proliferative agent.

9. A stent for implanting in a bodily lumen comprising a degradable structural element including:
an outer region above an inner region, the outer region including a first active agent and the inner region including a second active agent, wherein the inner region has a slower degradation rate than the outer region.

10. The stent of claim 9, wherein one of the active agents is an anti-inflammatory agent and the other active agent is an antiproliferative agent.

11. A stent for implanting in a bodily lumen comprising a degradable structural element, the structural element comprising:
an abluminal layer and a luminal layer, the abluminal layer having a different degradation rate than the luminal layer; and
a plurality of particles configured to treat a bodily disorder releasably embedded within at least one degrading layer, wherein the particles are configured to be released from the structural element due to erosion of the at least one layer during use of the stent.

12. The stent according to claim 11, wherein the abluminal layer has a faster degradation rate than the luminal layer so that the luminal layer maintains structural integrity of the stent as erosion of the abluminal layer allows particles to be released.

13. The stent according to claim 11, wherein the luminal layer has a faster degradation rate than the abluminal layer so that the abluminal layer maintains structural integrity of the stent as erosion of the luminal layer allows particles to be released.

14. The stent according to claim 11, wherein the particles in the abluminal layer have different treatment properties than the particles in the luminal layer.

15. The stent according to claim 11, wherein at least some of the particles comprise at least one type of active agent.

16. A stent for implanting in a bodily lumen comprising a biodegradable structural element, the structural element comprising:
an abluminal layer, a luminal layer, and an inner layer, the inner layer having a different degradation rate than the abluminal layer and the luminal layer; and
a plurality of particles releasably embedded within at least one layer, wherein the particles are configured to be released from the structural element due to erosion of the at least one layer during use of the stent, and the plurality of particles are configured to treat a bodily disorder.

17. The stent according to claim 16, wherein the inner layer has a faster degradation rate than the abluminal layer and the luminal layer so that the luminal layer and/or abluminal layers maintain structural integrity of the stent as erosion of the inner layer allows particles to be released.

18. The stent according to claim 16, wherein the inner layer has a slower degradation rate than the abluminal layer and the luminal layer so that the inner layer maintains structural integrity of the stent as erosion of the abluminal and/or luminal layers allow particles to be released.

19. A stent for implanting in a bodily lumen comprising a biodegradable structural element, the structural element comprising:
a proximal axial segment and a distal axial segment, the proximal axial segment having a different degradation rate than the distal axial segment.

20. A stent for implanting in a bodily lumen comprising a structural element, the structural element comprising:
a proximal axial segment and a distal axial segment, the proximal axial segment having a different degradation rate than the distal axial segment; and
a plurality of particles releasably embedded within at least one segment, the particles being configured to be released from the structural element due to erosion of the at least one segment during use of the stent, the plurality of particles being configured to treat a bodily disorder.

21. The stent according to claim 20, wherein the distal axial segment has a faster degradation rate than the proximal axial segment which allows a majority of the particles in the distal axial segment to be released before a majority of particles in the proximal axial segment.

22. The stent according to claim 20, wherein the plurality of particles in the proximal axial segment have different treatment properties than the plurality of particles in the distal axial segment.

23. The stent according to claim 20, wherein at least some of the plurality of particles comprise at least one type of active agent.

24. A stent for implanting in a bodily lumen comprising a structural element, the structural element comprising:
a proximal axial segment, a distal axial segment, and an inner axial segment, the inner axial segment having a different degradation rate than the proximal and/or distal axial segment.

25. The stent according to claim 24, further including an active agent in at least one segment.

26. The stent according to claim 24, further including an active agent within at least two segments, wherein the active agent in at least one segment is the same or different from the active agent within another segment.

27. A stent for implanting in a bodily lumen comprising a structural element, the structural element comprising:
a proximal axial segment, a distal axial segment, and an inner axial segment, the inner axial segment having a different degradation rate than the proximal and/or distal axial segment,
a plurality of particles releasably embedded within at least one segment, the particles being configured to be released from the structural element due to erosion of the at least one segment during use of the stent, the plurality of particles being configured to treat a bodily disorder.

28. The stent according to claim 27, wherein the inner segment has a faster degradation rate than the proximal axial segment and the distal axial segment which allows a majority of the particles in the inner axial segment to be released before a majority of particles in the proximal and distal axial segments.

29. The stent according to claim 27, wherein the inner axial segment has a slower degradation rate than the proximal axial segment and the distal axial segment which allows a majority of the particles in the distal and/or proximal axial segments to be released before a majority of particles in the inner axial segment.

30. A stent for implanting in a bodily lumen comprising a degradable structural element that includes an abluminal layer and a luminal layer, wherein at least one of the two layers comprise depots, the depots having a biodegradable material which at least partially fills the depots, the biodegradable material of the depots having a faster degradation rate than the layer.

31. The stent according to claim 30, wherein the depots comprise an active agent.

32. The stent according to claim 30, wherein the depots comprise particles.

33. The stent according to claim 32, wherein the particles in the depots comprise an active agent.
